(19) United States
(12) Patent Application Publication
(10) Pub. No.: US 2011/0281839 A1
(43) Pub. Date: Nov. 17, 2011

(54) COMBINATION THERAPY FOR THE TREATMENT OF BACTERIAL INFECTIONS

(76) Inventor: Janet PETERSON, Highlands, NJ (US)

(21) Appl. No.: 13/102,176

(22) Filed: May 6, 2011

Related U.S. Application Data
(60) Provisional application No. 61/345,209, filed on May 17, 2010.

(51) Int. Cl. A61K 31/407; A61P 31/04

(52) U.S. Cl. 514/210.12

(57) ABSTRACT

The present invention is directed to co-therapy and methods for the treatment of bacterial infections due to A. baumannii, such as pneumonia, blood stream infections, wound infections, urinary tract infection and intra-abdominal infections.

### CROSS-REFERENCE TO RELATED APPLICATIONS

The present application claims the benefits of the filing of U.S. Provisional Application No. 61/345,209 filed May 17, 2010. The complete disclosures of the aforementioned related patent applications are hereby incorporated herein by reference for all purposes.

### FIELD OF THE INVENTION

The present invention is directed to co-therapy and methods for the treatment of bacterial infections due to A. baumannii, such as pneumonia, blood stream infections, wound infections, urinary tract infection and intra-abdominal infections.

### BACKGROUND OF THE INVENTION

A. baumannii, a gram negative bacteria which causes pneumonia, is a multiple drug resistant bacteria which can resist nearly all currently available antibiotics (GOOTZ, T. D., “The Global Problem of Antibiotic Resistance”, Crit Rev Immunol., 2010, pp79-93, Vol. 30). As a result of its resistance to drug treatment, some estimates state the A. baumannii infections, including severe pneumonia, infections of the urinary tract, bloodstream and other parts of the body, kill tens of thousands of U.S. hospital patients each year. Antimicrobial agents currently available which can be used to treat some cases of infection due to A. baumannii include sulbactam, cabapenems, aminoglycosides, tigecycline and polymixin-based compounds such as colistin. However, many of the antimicrobial agents of last resort, such as colistin, may be toxic to the patient.

First line treatment for A. baumannii infections is a carbapenem antibiotic such as imipenem, meropenem, and the like (although carbapenem resistance is increasingly common). Doripenem is a carbpenem antimicrobial agent with activity against A. baumannii. Patients with pneumonia due to A. baumannii with minimum inhibitory concentrations (MICs) as high as 16 pg/mL have been shown to be effectively treated with doripenem given at a dosage of 1 gram (g) over a 4 hour infusion (AMBRUZS, M., et al., “ Clinical Outcome of Nosocomial Pneumonia (NP) / Ventilator-Associated Pneumonia (VAP), or Health-Care Associated Pneumonia (HCAP) After Treatment with Doripenem 1 g Infused over 4 Hours every 8 Hours in a Study Protocol that Enriched for Infection with P. aeruginosa (Psa)”, Poster presented at the 49th lnterscience Conference on Antimicrobial Agents and Chemotherapy (ICAAC); September 12-15, 2009; San Francisco, California, USA (Poster K-293)). Other treatment options for A. baumannii infections include antibiotics such as polymixins, tigecycline and aminoglycosides.

In vitro synergy testing and animal models have suggested that combinations of doripenem and colistin, ampicillin/sulbactam, levofloxacin, or combinations of polymixin B, and rifampin may be synergistic for the treatment of multidrug resistant (MDR) A. baumannii infections (SHIELDS, R.K., et al.

“Increased Resistance to Tigecycline Aming Pan-Resistant Acinetobacter: The Value of a Carbapenem in Combination with Colistin or Sulbactam”, Poster presented at the 49th lnterscience Conference on Antimicrobial Agents and Chemotherapy (ICAAC); September 12-15, 2009; San Francisco, California, USA (Poster E-1458); SHIELDS, R.K., et al., “Microbiologic and Clinical Evidence Supporting the Combination of Doripenem and Colistin for the Treatment of Pan-resistant Acinetobacter”, Poster presented at the 49th lnterscience Conference on Antimicrobial Agents and Chemotherapy (ICAAC); September 12-15, 2009; San Francisco, California, USA (Poster E-1459); BANTAR, C., et al., “Comparative time-kill study of doxycycline, tigecycline, sulbactam, and imipenem against several clones of Acinetobacter baumannii”, Diagn Microbiol Infect Dis., 2008, pp 309-314, Vol. 61; PANKUCH, G.A., et al., “Activity of Doripenem With and Without Levofloxacin, Amikacin, and Colistin Against Acinetobacter baumannii by Synergy Time-Kill”, Poster presented at the 49th lnterscience Conference on Antimicrobial Agents and Chemotherapy (ICAAC); September 12-15, 2009; San Francisco, California, USA (Poster E-1446); URBAN, C., et al., :In Vitro Double and Triple Synergistic Activities of Doripenem, Polymixin B, and Rifampin Against Multidrug-Resistant Acinetobacter baumannii, Pseudomonas aeruginosa, Klebsiella pneumonia and Escherichia coli”, 49^{th} ICAAC, 2009; Poster E-1447; PANKUCH, G.A., et al., “Activity of doripenem with and without levofloxacin, amikacin and colistin against Pseudomonas aeruginosa and Acinetobacter baumannii”, Diagn Microbiol Infect Dis., 2010 Mar 23. [Electronic publication ahead of print]; STROUP, J. S., et al., “Novel Treatment approach to combat an infection with Acinetobacter”, Proc (Baylor Univ Med Cent)., 2010, pp29-30, Vol. 23; and PONGPECH, P., et al., “Antibacterial Activity of Carbapenem-Based Combinations Against Multidrug Resistant Acinetobacter baumannii”, J. Med. Accos. Thai., 2010, pp 161-171, Vol. 93, No. 2). Limited in vivo data in the form of case reports have demonstrated that doripenem along with colistin, or a combination of colistin and tigecycline, is effective in treating pneumonia due to A. baumannii (GOOTZ, T. D., “The Global Problem of Antibiotic Resistance”, Crit Rev Immunol., 2010, pp79-93, Vol. 30; and PANKUCH, G.A., et al., “Activity of doripenem with and without levofloxacin, amikacin and colistin against Pseudomonas aeruginosa and Acinetobacter baumannii”, Diaqn Microbiol Infect Dis., 2010 Mar 23. [Electronic publication ahead of print]) However, there remains a need to identify combinations of antimicrobial agents, including combinations with doripenem, which would be effective and preferably synergistic, for the treatment of A. baumannii bacterial infections, including but not limited to pneumonia.

### SUMMARY OF THE INVENTION

The present invention is directed to methods of co-therapy for the treatment of bacterial infections due to A. baumannii, comprising administering to a subject in need thereof a therapeutically effective amount of co-therapy comprising (a) doripenem and (b) a fluoroquinolone antimicrobial agent. In an embodiment of the present invention, the fluoroquinolone antimicrobial agent is selected from the group consisting ciprofloxacin and levofloxacin.

The present invention is further directed to a pharmaceutical composition comprising (a) doripenem, (b) a fluoroquinolone antimicrobial agent, preferably a fluoroquinolone selected from the group consisting ciprofloxacin and levofloxacin, and (c) a pharmaceutically acceptable carrier. An illustration of the invention is a pharmaceutical composition made by mixing comprising (a) doripenem, (b) a fluoroquinolone antimicrobial agent, preferably a fluoroquinolone selected from the group consisting ciprofloxacin and levofloxacin, and (c) a pharmaceutically acceptable carrier. Illustrating the invention is a process for making a pharmaceutical composition comprising mixing comprising (a) doripenem, (b) a fluoroquinolone antimicrobial agent, preferably a fluoroquinolone selected from the group consisting ciprofloxacin and levofloxacin, and (c) a pharmaceutically acceptable carrier.

### DETAILED DESCRIPTION OF THE INVENTION

The present invention is directed to methods for the treatment of bacterial infections due to A. baumannii comprising administering to a subject in need thereof a therapeutically effective amount of co-therapy comprising (a) doripenem and (b) a fluoroquinolone antimicrobial agent, preferably a fluoroquinolone selected from the group consisting ciprofloxacin and levofloxacin.

In an embodiment of the present invention, the bacterial infection due to A. baumannii is selected from the group consisting of pneumonia, blood stream infection, wound infection, urinary tract infection and intra-abdominal infection, preferably pneumonia.

In an embodiment, the present invention is directed to methods of co-therapy for the treatment of pneumonia due to A. baumannii. In another embodiment, the present invention is directed to methods of co-therapy for the treatment of pneumonia due to A. baumannii, wherein the A. baumanii is non-susceptible to doripenem. In another embodiment, the present invention is directed to methods of co-therapy for the treatment of pneumonia due to A. baumannii, wherein the A. baumanii is non-susceptible to doripenem and further non-susceptible to the selected fluorquinolone, preferably ciprofloxacin or levofloxacin.

As used herein, unless otherwise noted, the term “carbapenem antimicrobial agent” shall mean any of the class of beta-lactam antibiotics with a broad spectrum of antibacterial activity. As a result of their structure, carbapenem antimicrobial agents are highly resistant to beta-lactamases. Suitable examples of carbapenem antimicrobial agents include, but are not limited to imipenem, meropenem, ertapenem, doripenem, panipenem/betamipron, biapenem and PZ-601 (a carbapenem antibiotic currently under clinical testing). Preferably, the carbapenem antimicrobial agent is selected from the group consisting of imipenem, meropenem and doripenem. More preferably, the carbapenem antimicrobial agent is doripenem.

As used herein, unless otherwise noted, the term “doripenem” shall mean doripenem monohydrate, a broad spectrum injectable antibiotic, also known by, for example, the brand names DORIBAX, FINIBAX, and other.

As used herein, unless otherwise noted, the term “fluoroquinolone antimicrobial agent” shall mean any of the broad spectrum antimicrobial agents against gram-positive and gram-negative bacteria, characterized by the presence of a fluorine substituted quinolone within its structure Suitable examples include, but are not limited to, alatrofloxacin, balofloxacin, ciprofloxacin, clinafloxacin, danofloxacin, delafloxacin, difloxacin, enoxacin, gatifloxacin, gemifloxacin, grepafloxacin, loevofoxacin, lomefloxacin, marbofloxacin, ofloxacin, orbifloxacin, pefloxacin, sirafloxacin, sparfloxacin, temafloxacin, tosufloxacin and trovafloxacin. Preferably, the fluoroquinolone is selected from the group consisting of ciprofloxacin and levofloxacin.

As used herein, unless otherwise noted, the term “colistin” shall mean COLISTIN POLYXIN E, a polymixin antibiotic produced by certain strains of Bacillus polymyxa var. colistinus, effective against most gram-negative bacilli and further used as a last resort antibiotic against multidrug resistant pseudomonas aeruginosa and acinetobacter.

As used herein, unless otherwise noted, the term “amikacin” shall mean an aminoglycoside antibiotic which works by binding to the bacterial 30S ribosomal subunit, causing misreading of mRNA and leaving the bacterium unable to synthesize proteins vital for growth. Amikacin is useful for the treatment of severe, hospital-acquired infections with multidrug resistant gram-negative bacteria such as Pseudomonas aeruginosa, Acinetobacter spp. and Enterobacter spp.

As used herein, unless otherwise noted, the term “tobramycin” shall mean tobranycin sulfate, an aminoglycoside antibiotic used to treat gram-negative bacterial infections such as Pseudomonas aeruginosa and Acinetobacter spp.

One skilled in the art can readily determine dosages and regimens for the administration of the carbapenem (preferably doripenem) and fluoroquinolone (preferably ciprofloxacin or levofloxacin) antimicrobial agents of the co-therapy of the present invention, for example by consulting the PDR (Physician's Desk Reference) and / or the FDA required drug literature included with the pharmaceutical agent. For example, a representative dosage for doripenem is 500 mg every 8 hours or 1 g every 8 hours; for ciprofloxacin is 400 mg every 12 hours (IV) or 500 mg every 12 hours (oral); and for levofloxacin is 250 mg once, 500 mg once daily or 750 mg once daily.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound or co-therapy of the present invention to prevent the onset of the symptoms or complications, alleviate the symptoms or complications, or eliminate the disease, condition, or disorder.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes prevention of the onset of the symptoms or complications, alleviation of the symptoms or complications, or elimination of the disease, condition, or disorder.

Wherein the present invention is directed to co-therapy or combination therapy, comprising administration of (a) doripenem and (b) a fluoroquinolone antimicrobial agent, “therapeutically effective amount” shall mean that amount of the combination of agents taken together so that the combined effect elicits the desired biological or medicinal response. For example, the therapeutically effective amount of co-therapy comprising administration of a) doripenem and (b) a fluoroquinolone antimicrobial agent, would be the amount of (a) doripenem and (b) a fluoroquinolone antimicrobial agent, that when taken together or sequentially have a combined effect that is therapeutically effective. Further, it will be recognized by one skilled in the art that in the case of co-therapy with a therapeutically effective amount, as in the example above, the amount of the (a) doripenem and/or the amount of the (b) fluoroquinolone antimicrobial agent individually may or may not be therapeutically effective.

As used herein, the terms “co-therapy” and “combination therapy” shall mean treatment of a subject in need thereof by administering (a) doripenem and (b) a fluoroquinolone antimicrobial agent, wherein the (a) doripenem, the (b) fluoroquinolone antimicrobial agent are administered by any suitable means, simultaneously, sequentially, separately or in a single pharmaceutical dosage form. Where the (a) doripenem and the (b) fluoroquinolone antimicrobial agent are administered in separate dosage forms, the number of dosages administered per day for each compound may be the same or different. The (a) doripenem and the (b) fluoroquinolone antimicrobial agent may be administered via the same or different routes of administration. Examples of suitable methods of administration include, but are not limited to, oral, intravenous (iv), intramuscular (im), subcutaneous (sc), transdermal, and rectal. The (a) doripenem and/or (b) fluoroquinolone antimicrobial agent may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The (a) doripenem and the (b) fluoroquinolone antimicrobial agent may be administered according to simultaneous or alternating regimens, at the same or different times during the course of the therapy, concurrently in divided or single forms.

As used herein, the term “composition” is intended to encompass a product comprising the specified ingredients in the specified amounts, as well as any product which results, directly or indirectly, from combinations of the specified ingredients in the specified amounts.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The present invention further comprises pharmaceutical compositions containing (a) doripenem, (b) a fluoroquinolone antimicrobial agent and (c) a pharmaceutically acceptable carrier. Pharmaceutical compositions of the present invention described herein can be prepared by intimately mixing the (a) doripenem and the (b) fluoroquinolone antimicrobial agent with a pharmaceutical carrier according to conventional pharmaceutical compounding techniques.

The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include diluents, granulating agents, lubricants, binders, disintegrating agents, drug release controlling hydrophilic polymer, drug release controlling hydrophobic polymers, wetting agents and the like. Solid oral preparations may also be coated with substances such as sugars, cellulosic ethers, and acrylic polymers for extended release or may be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, the (a) doripenem and the (b) fluoroquinolone antimicrobial agent, as the active ingredients, are intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, and tablets (including caplets), suitable carriers and additives include diluents, granulating agents, lubricants, binders, disintegrating agents, drug release controlling hydrophilic polymers, drug release controlling or hydrophobic polymers, wetting agents, and the like. Because of their ease in administration, tablets and capsules represent the most advantageous oral dosage unit form, in which case solid pharmaceutical carriers are obviously employed. If desired, tablets may be sugar coated or may be enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions described herein will contain, per dosage unit, e.g., tablet, capsule, powder, injection, teaspoonful and the like, an amount of the active ingredient(s) necessary to deliver an effective dose as described above. The pharmaceutical compositions herein will contain, per unit dosage unit, e.g., tablet, capsule, powder, injection, suppository, teaspoonful and the like, of from about 0.01 to about 2,000 mg, or any amount or range therein (for example 250 mg, 400 mg, 500 mg, 750 mg, 1000 mg, and the like), independently, of each of the (a) doripenem and the (b) fluoroquinolone antimicrobial agent. The pharmaceutical compositions described herein may be given at a suitably selected therapeutically effective dosage, which may be varied depending upon the requirement of the patients, the severity of the condition being treated and the compound being employed. The use of either daily administration or post-periodic dosing may be employed.

Preferably these pharmaceutical compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the pharmaceutical composition may be presented in a form suitable for once-weekly or once-monthly administration; for example, an insoluble salt of the active compound(s), such as the decanoate salt, may be adapted to provide a depot preparation for intramuscular injection. For preparing solid compositions such as tablets, the principal active ingredient(s) are mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid formulation composition containing a mixture of the active ingredient(s). The tablets or pills of the pharmaceutical composition of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the pharmaceutical compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

Advantageously, the pharmaceutical compositions of the present invention may be administered in a single daily dose, or the total daily dosage may be administered in divided doses of two, three or four times daily. Furthermore, the pharmaceutical compositions of the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

In certain embodiments, for oral administration in the form of a tablet or capsule, the active drug component(s) can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride, and the like. Disintegrators include, without limitation, starch, sodium starch glycolate, croscamellose sodium, crospovidone, methyl cellulose, agar, bentonite, xanthan gum, and the like. The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The co-therapy comprising (a) doripenem and (b) a fluoroquinolone antimicrobial agent, of the present invention, may be administered in any of the foregoing compositions and according to dosage regimens established in the art whenever treatment of a glucose related disorder is required.

Optimal dosages to be administered may be readily determined by those skilled in the art, and will vary with for example, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound or co-therapy to treat or prevent a given disorder. One skilled in the art will further recognize that human clinical trials including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

The unexpected synergistic effect of co-therapy of doripenem and a fluoroquinolone antimicrobial agent is based on analysis of combination therapy in human patients, as described in more detail in Example 1.

Example 1

Adult subjects with nosocomial pneumonia enrolled in 3 clinical studies of doripenem who had A. baumannii isolated at study entry were identified and assessed for clinical and microbiological outcomes at the test of cure visit (TOC). The subjects/outcomes for the patients were analyzed according to the test groups, as indicated in Table 1, below. Each group received treatment of either doripenem alone or doripenem with concomitant therapy, as noted.

TABLE 1
Treatment Groups

| Treatment/Combination | No. of Patients |
|---|---|
| Doripenem Monotherapy | 9 |
| Doripenem + Fluoroquinolone | 4 |
| Doripenem + Colistin | 3 |
| Doripenem + Amikacin | 9 |
| Doripenem + Tobramycin | 6 |

True concomitant therapy was determined by applying the following algorithm: treatment with the second antimicrobial was initiated during Study Days 1-14 and administered on the same study days as doripenem. In addition, the second antimicrobial was given for >1 day of the overlapping period. Concomitant therapy was assessed for the following antimicrobial agents: the fluoroquinolones ciprofloxacin and levofloxacin, colistin and the aminoglycosides amakacin and tobramycin.

Doripenem was administered at either 500 mg Q8H as a 1 hour infusion, 500 mg Q8H as a 4 hour infusion or 1g Q8H as a 4 hour infusion, depending on the specific clinical study, as noted in the Tables below. The dosage regimen for the concominant antimicrobial agent was as listed in the Tables below.

Susceptibility of the A. baumannii isolates to each of the agents was determined by broth microdilution using Clinical and Laboratory Standards Institute (CLSI) methodology at a central laboratory. A. baumannii was considered susceptible (S) to doripenem if the measured minimum inhibitory concentration (MIC) was pg/mL and nonsusceptible (NS) if the MIC 2pg/rnL. The susceptibility of A. baumannii to other drugs was interpreted according to the standard procedures as defiend by CLSI (Clinical and Laboratory Standards Institute Test Procedures MO2-A10, Vol. 29, No. 1; M07-A8, Vol. 29, No. 2 and/or M100-S19, Vol. 29, No. 3; Jan. 2009).

Tables 2-6, below list the results for each patient and test group. Susceptibility is noted in parentheses in the treatment MIC column. As indicated in the data below, doripenem monotherapy yielded a 7/9 (77.8%) positive outcome. By comparison, co-therapy with doripenem in combination with a fluoroquinolone selected from the group consisting of ciprofloxacin and levofloxacin yielded a 4/4 (100%) positive outcome; doripenem in combination with colistin yielded a 2/3 (66.7%) positive outcome; doripenem in combination with amikacin yielded a 6/9 (66.7%) positive outcome; and doripenem in combination with tobramycin yielded a 4/6 (66%) positive outcome.

TABLE 2
Doripenem Monotherapy Results

| | | | | Micro- |
|---|---|---|---|---|
| | Doripenem MIC | | Clinical | biological |
| Patient | (μg/mL) | | Outcome | Outcome |
| 1 | >128 | (NS) | Failure | Persisted |
| 2 | 8 | (NS) | Failure | Eradicated |
| 3 | 1 | (S) | Cure | Eradicated |
| 4 | 0.25 | (S) | Cure | Eradicated |
| 5 | 0.25 | (S) | Cure | Eradicated |
| 6 | 0.12 | (S) | Cure | Eradicated |
| 7 | 0.12 | (S) | Cure | Eradicated |
| 8 | 0.5 | (S) | Cure | Eradicated |
| 9 | 0.5 | (S) | Cure | Eradicated |

Doripenem dosed 1 gram every 8 hours or 500 mg every 8 hours
Doripenem: Susceptible (S) ≦1 μg/mL, Non-susceptible (NS) >2 μg/mL

TABLE 3
Doripenem + Fluoroquinolone Results

| | | | | | | Micro- |
|---|---|---|---|---|---|---|
| | Doripenem | | Fluoroquinolone | | Clinical | biological |
| Patient | MIC (μg/mL) | | MIC (μg/mL) | | Outcome | Outcome |
| | | | Ciprofloxacin | | | |
| 1 | >32 | (NS) | 8 | (NS) | Cure | Eradicated |
| | | | Levofloxacin | | | |
| 2 | >32 | (NS) | >4 | (NS) | Cure | Eradicated |
| 3 | 32 | (NS) | >4 | (NS) | Cure | Eradicated |
| 4 | 16 | (NS) | >4 | (NS) | Cure | Eradicated |

Doripenem dosed 1 gram every 8 hours or 500 mg every 8 hours;
Ciprofloxacin dosed at 400 mg IV, twice daily; Levofloxacin dosed at 750 mg once daily or 500 mg once daily
Ciprofloxacin: Susceptible (S) ≦1 μg/mL, Non-susceptible (NS) ≧2 μg/mL;
Levofloxacin: Susceptible (S) ≦2 μg/mL, Non-susceptible (NS) ≧4 μg/mL
Doripenem: Susceptible (S) ≦1 μg/mL, Non-susceptible (NS) >2 μg/mL

TABLE 4
Doripenem + Colistin Results

| | | | | | Micro- |
|---|---|---|---|---|---|
| | Doripenem MIC | | Colistin MIC | Clinical | biological |
| Patient | (μg/mL) | | (μg/mL) | Outcome | Outcome |
| 1 | >32 | (NS) | 1 (S) | Cure | Eradicated |
| 2 | 32 | (NS) | 1 (S) | Cure | Eradicated |
| 3 | 1 | (S) | 1 (S) | Failure | Eradicated |

Doripenem dosed 500 mg every 8 hours or 1 gram every 8 hours; Colistin dosed according to manufacturer's instructions
Colistin: Susceptible (S) ≦2 μg/mL, Non-susceptible (NS) ≧4 μg/mL
Doripenem: Susceptible (S) ≦1 μg/mL, Non-susceptible (NS) >2 μg/mL

TABLE 5
Doripenem + Amikacin Results

| | | | | | | Micro- |
|---|---|---|---|---|---|---|
| | Doripenem MIC | | Amikacin MIC | | Clinical | biological |
| Patient | (μg/mL) | | (μg/mL) | | Outcome | Outcome |
| 1 | 64 | (NS) | >64 | (NS) | Cure | Eradicated |
| 2 | >32 | (NS) | >64 | (NS) | Failure | Persisted |
| 3 | 16 | (NS) | >64 | (NS) | Cure | Eradicated |
| 4 | 64 | (NS) | >64 | (NS) | Cure | Eradicated |
| 5 | 1 | (S) | >64 | (NS) | Failure | Persisted |
| 6 | 1 | (S) | 4 | (S) | Cure | Eradicated |
| 7 | 0.25 | (S) | 1 | (S) | Cure | Eradicated |
| 8 | 0.12 | (S) | 1 | (S) | Cure | Eradicated |

| | | | | | |
|---|---|---|---|---|---|
| 9 | Unknown | 2 | (S) | Failure | Persisted |

Doripenem dosed 1 gram every 8 hours or 500 mg every 8 hours; Amikacin dosed according to renal function per manufacturer's instructions
Amikacin: Susceptible (S) ≦16 μg/mL, Non-susceptible (NS) ≧32 μg/mL;
Doripenem: Susceptible (S) ≦1 μg/mL, Non-susceptible (NS) >2 μg/mL

TABLE 6
Doripenem + Tobramycin Results

| | | | | | | Micro- |
|---|---|---|---|---|---|---|
| | Doripenem MIC | | Tobramycin | | Clinical | biological |
| Patient | (μg/mL) | | MIC (μg/mL) | | Outcome | Outcome |
| 1 | >32 | (NS) | Unknown | | Failure | Persisted |
| 2 | 1 | (S) | 4 | (S) | Cure | Eradicated |
| 3 | 0.25 | (S) | 0.25 | (S) | Cure | Eradicated |
| 4 | 0.12 | (S) | Unknown | | Cure | Eradicated |
| | 0.12 | (S) | 0.5 | (S) | Failure | Persisted |
| | 0.12 | (S) | 0.5 | (S) | Cure | Eradicated |

Doripenem dosed 1 gram every 8 hours or 500 mg every 8 hours; Tobramycin dosed according to manufacturer's instructions
Tobramycin: Susceptible (S) ≦4 μg/mL, Non-susceptible (NS) ≧8 μg/mL;
Doripenem: Susceptible (S) ≦1 μg/mL, Non-susceptible (NS) >2 μg/mL

An analysis of the above presented results demonstrates the following treatment effects:

(a) For the treatment of nosocomial pneumonia due to doripenem-susceptible A. baumannii, doripenem monotherapy was effective.

(b) For infections due to doripenem non-susceptible A. baumannii, doripenem in combination with colistin appeared to be effective. However, the isolates were susceptible to colistin; thus, it is unclear whether the efficacy observed was due to colistin alone or synergy between the two antibiotics.

(c) For infections due to A. baumannii that was resistant (non-susceptible) to both agents, the combination of doripenem and a fluoroquinolone was effective, with 4/4 (100%) subjects cured, indicating a synergistic effect. Thus, for infections due to multiple-drug resistant A. baumannii, co-therapy with doripenem and a fluoroquinolone such as ciprofloxacin or levofloxacin, may be more effective and less toxic than treatment with colistin-based regimens.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method for treating a bacterial infection due to A. baumannii comprising administering to a subject in need thereof a therapeutically effective amount of co-therapy comprising (a) doripenem; and (b) a fluoroquinolone antimicrobial agent.

2. A method as in claim 1, wherein the fluoroquinolone antimicrobial agent is selected from the group consisting of ciprofloxacin and levofloxacin.

3. A method as in claim 1, wherein the bacterial infection due to A. baumannii is selected from the group consisting of pneumonia, a blood stream infection, a wound infection, a urinary tract infection and an intra-abdominal infection.

4. A method as in claim 3, wherein the bacterial infection due to A. baumannii is pneumonia.

5. A method as in claim 1, wherein the A. baumannii is non-susceptible to doripenem.

6. A method as in claim 1, wherein the A. baumannii is non-susceptible to doripenem and is further non-susceptible to the fluoroquinolone antimicrobial agent.
